(19) United States
(12) Patent Application Publication
(10) Pub. No.: US 2026/0027178 A1
(43) Pub. Date: Jan. 29, 2026

(54) Treatment Of Parkinson's Disease With SIM BHLH Transcription Factor 2 (SIM2) Agonists

(71) Applicant: Regeneron Pharmaceuticals, Inc., Tarrytown, NY (US)

(72) Inventors: Sahar Gelfman, Tarrytown, NY (US); Pascaline Aime-Wilson, Tarrytown, NY (US); Eli Stahl, Tarrytown, NY (US); Rujin Wang, Tarrytown, NY (US); Chitra Suri, Tarrytown, NY (US); Kevin Kanning, Tarrytown, NY (US); Ilias Pavlopoulos, Tarrytown, NY (US); Giovanni Coppola, Tarrytown, NY (US); Yusuke Fukuda, Tarrytown, NY (US)

(21) Appl. No.: 19/280,514

(22) Filed: Jul. 25, 2025

Related U.S. Application Data
(60) Provisional application No. 63/676,078, filed on Jul. 26, 2024.

(51) Int. Cl. A61K 38/17; A61K 31/341; A61K 31/415; A61K 31/42; A61K 31/444; A61K 45/06; C12Q 1/6883

(52) U.S. Cl. CPC A61K 38/17; A61K 31/341; A61K 31/415; A61K 31/42; A61K 31/444; A61K 45/06; C12Q 1/6883; C12Q 2600/156

(57) ABSTRACT

The present disclosure generally relates to the treatment of subjects having Parkinson's disease or at risk of developing Parkinson's disease by administering a SIM BHLH Transcription Factor 2 (SIM2) agonist to the subject.

### FIELD

The present disclosure generally relates to the treatment of subjects having Parkinson's disease or at risk of developing Parkinson's disease, by administering a SIM BHLH Transcription Factor 2 (SIM2) agonist to the subject, and to methods of identifying subjects having an increased risk of developing Parkinson's disease.

### BACKGROUND

Parkinson's disease (PD), or simply Parkinson's, is a chronic degenerative disorder of the central nervous system that affects both the motor system and non-motor systems. The pathology of Parkinson disease involves the loss of dopaminergic neurons in the substantia nigra and the presence of Lewy bodies (intraneuronal accumulations of aggregated proteins), in surviving neurons in various areas of the brain. The disease is progressive and usually manifests after the age of 50 years, although early-onset cases (before 50 years) are known. The majority of Parkinson's cases are sporadic, suggesting a multifactorial etiology based on environmental and genetic factors. Symptoms begin gradually, often on one side of the body, and later affecting both sides. Symptoms include trembling of hands, arms, legs, jaw and face, stiffness of the arms, legs and trunk, slowness of movement, and poor balance and coordination. As symptoms get worse, people with the disease may have trouble walking, talking, or doing simple tasks. Parkinson's patients may also have problems such as depression, sleep problems, or trouble chewing, swallowing, or speaking. There is no cure for the disease and its exact cause is not known, but there are effective treatments that can relieve symptoms.

SIM BHLH Transcription Factor 2 (SIM2) (also known as transcription factor single-minded 2) is encoded by a 50 kb gene located at 21q22.13. SIM2 protein is 667 amino acids long and is a 73 kDa transcription factor that is a regulator of neurogenesis. Two isoforms (long and short) of SIM2 exist (Sanchez et al., Cell Death Differ., 2023, 30, 1472-1487). The SIM2 protein is ubiquitinated by RING-IBR-RING-type E3 ubiquitin ligases, including the parkin RBR E3 Ubiquitin Protein Ligase (PRKN). This gene maps within the so-called Down syndrome chromosomal region and may contribute to some specific Down syndrome phenotypes. A recent study shows SIM2 directly mediates PRKN-dependent mitophagy in mice mammary epithelial cells (Sanchez et al., Cell Death Differ., 2023, 30, 1472-1487).

### SUMMARY

The present disclosure provides methods of treating a subject having Parkinson's disease, or at risk of developing Parkinson's disease, the methods comprising administering a SIM2 agonist to the subject.

The present disclosure also provides methods of treating a subject having Parkinson's disease or at risk of developing Parkinson's disease by administering a Parkinson's disease therapeutic agent, the methods comprising: determining or having determined whether the subject has a SIM2 variant nucleic acid molecule, by: obtaining or having obtained a biological sample from the subject; and performing or having performed a sequence analysis on the biological sample to determine if the subject has a genotype comprising a SIM2 variant nucleic acid molecule; and administering or continuing to administer the Parkinson's disease therapeutic agent in an amount that is the same as a standard dosage amount to a subject that is SIM2 reference; or administering or continuing to administer the Parkinson's disease therapeutic agent in an amount that is the same as or less than a standard dosage amount, and/or administering a SIM2 agonist to a subject that is heterozygous or homozygous for the SIM2 variant nucleic acid molecule; wherein the presence of the SIM2 variant nucleic acid molecule indicates the subject has an increased risk of developing Parkinson's disease.

The present disclosure also provides methods of identifying a subject having an increased risk of developing Parkinson's disease, the methods comprising: determining or having determined the presence or absence of a SIM2 variant nucleic acid molecule in a biological sample obtained from the subject; wherein: when the subject is SIM2 reference, then the subject has a decreased risk of developing Parkinson's disease; and when the subject is heterozygous or homozygous for the SIM2 variant nucleic acid molecule, then the subject has an increased risk of developing Parkinson's disease.

The present disclosure also provides Parkinson's disease therapeutic agents for use in the treatment or prevention of Parkinson's disease in a subject having a SIM2 variant nucleic acid molecule.

The present disclosure also provides SIM2 agonists for use in the treatment or prevention of Parkinson's disease in a subject that is heterozygous or homozygous for a SIM2 variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Western blot analysis of SIM2 protein levels showing that SIM2 can be detected in lysates from different cell types and from mouse and human brain tissues; from left: HepG2 human liver cancer cell line; SHSY5Y neuroblastoma cell line; mouse primary cortical neuron; human brain tissue from cortex BA4; mouse brain tissue with ventral midbrain dissected out (ROB: rest of brain); and mouse ventral midbrain tissue (Mid); actin protein levels are shown as a loading control.

FIG. 2 shows knockdown of SIM2 protein and mRNA by shRNA validates specificity of the antibody.

FIG. 3 (Panels A, B, and C) shows a rare pLoF mask and increasing risk of PD; risk arising from loss of SIM2 is maintained in broad and strict Parkinson's disease definitions (Panel A); more common pLoF masks maintain risk with same case carriers (Panel B); and missense only masks (top) do not show risk-pointing to a clear loss of function mechanism (Panel C).

FIG. 4 (Panels A, B, and C) shows eQTLs increasing SIM2 show protection from PD; top eQTLs increasing SIM2 expression from brain tissues (Panel A); Metabrain eQTLs are consistent-decreasing SIM2 shows nominal risk (cerebellum), increasing SIM2 shows protection (cortex) (Panel B); and eQTLs increasing SIM2 shows nominal protection from PD (Panel C).

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that rare SIM2 variant nucleic acid molecules (rare pLoF mask, strict PD, AF<0.1%; OR=12.14; p=1.5e-07) (whether these variants are homozygous or heterozygous in a particular subject) associate with an increased risk of developing Parkinson's disease. It is believed that SIM2 variant nucleic acid molecules have not been associated with Parkinson's disease in humans. Therefore, subjects that are heterozygous or homozygous for a SIM2 variant nucleic acid molecule may be treated with a SIM2 agonist such that Parkinson's disease is inhibited or prevented, the symptoms thereof are reduced or prevented, and/or development of symptoms is repressed or prevented. It is also believed that such subjects having Parkinson's disease may further be treated with one or more Parkinson's disease therapeutic agents that treat or inhibit Parkinson's disease. In addition, the present disclosure provides methods of leveraging the presence or absence of SIM2 variant nucleic acid molecules in subjects to identify or stratify risk in such subjects of developing Parkinson's disease, or to diagnose subjects as having an increased risk of developing Parkinson's disease.

For purposes of the present disclosure, any particular subject, such as a human, can be categorized as having one of three SIM2 genotypes: i) SIM2 reference; ii) heterozygous for a SIM2 variant nucleic acid molecule; or iii) homozygous for a SIM2 variant nucleic acid molecule. A subject is SIM2 reference when the subject does not have a copy of a SIM2 variant nucleic acid molecule. A subject is heterozygous for a SIM2 variant nucleic acid molecule when the subject has a single copy of a SIM2 variant nucleic acid molecule. A subject is homozygous for a SIM2 variant nucleic acid molecule when the subject has two copies of a SIM2 variant nucleic acid molecule.

In any of the embodiments described herein, the SIM2 variant nucleic acid molecule can be any nucleic acid molecule (such as, a genomic nucleic acid molecule, an mRNA molecule, or a cDNA molecule produced from an mRNA molecule) encoding a SIM2 variant polypeptide having a partial loss-of-function, a complete loss-of-function, a predicted partial loss-of-function, or a predicted complete loss-of-function. A subject who has a SIM2 polypeptide having a partial loss-of-function (or predicted partial loss-of-function) is hypomorphic for SIM2. In some embodiments, the SIM2 variant nucleic acid molecule results in decreased or aberrant expression or activity of SIM2 mRNA or polypeptide. In some embodiments, the SIM2 variant nucleic acid molecule is associated with a reduced in vitro response to SIM2 ligands compared with reference SIM2. In some embodiments, the SIM2 variant nucleic acid molecule is a splice-site variant, a stop-gain variant, a start-loss variant, a stop-loss variant, a frameshift variant, an in-frame indel variant, or a variant that encodes a truncated SIM2 variant polypeptide. In some embodiments, the SIM2 variant nucleic acid molecule is a missense variant nucleic acid molecule. In some embodiments, the SIM2 variant nucleic acid molecule comprises a single nucleotide polymorphism (SNP). In some embodiments, the SIM2 variant nucleic acid molecule comprises a variation in a coding region. In some embodiments, the SIM2 variant nucleic acid molecule does not comprise a variation in a non-coding region, except for a splice acceptor region (two bases before the start of any exon except the first). In some embodiments, the SIM2 variant nucleic acid molecule results or is predicted to result in a premature truncation of a SIM2 polypeptide compared to the reference SIM2. In some embodiments, the SIM2 variant nucleic acid molecule is a variant that is predicted to be damaging to the protein function (and hence, in this case, protective to the human) by in vitro prediction algorithms such as Polyphen, SIFT, or similar algorithms. In some embodiments, the SIM2 variant nucleic acid molecule is a variant that causes or is predicted to cause a nonsynonymous amino acid substitution in a SIM2 nucleic acid molecule and whose allele frequency is less than 1/100 alleles in the population from which the subject is selected. In some embodiments, the SIM2 variant nucleic acid molecule is any rare missense variant (allele frequency <0.1%; or 1 in 1,000 alleles), or any splice-site, stop-gain, start-loss, stop-loss, frameshift, or in-frame indel, or other frameshift SIM2 variant.

In any of the embodiments described herein, the SIM2 variant genomic nucleic acid molecule may include one or more variations at any of the positions of chromosome 21 (i.e., positions 36,699,115-36,749,917) using the nucleotide sequence of the SIM2 reference genomic nucleic acid molecule in the GRCh38/hg38 human genome assembly (see, ENSG00000159263.16, ENST00000290399.11 annotated in the in the Ensembl database (URL: world wide web at “http://useast.ensembl.org/Homo_sapiens/Gene/Summary?g=ENSG00000159263;r=21:36699115-36749917;transcript=ENST00000290399.11”)) as a reference sequence. The sequences provided in these transcripts for the SIM2 genomic nucleic acid molecule are only exemplary sequences. Other sequences for the SIM2 genomic nucleic acid molecule are also possible.

In any of the embodiments described herein, the SIM2 variant nucleic acid molecule may comprise any one or more of the following genetic variations in the genomic nucleic acid molecule (referring to the chromosome: positions set forth in the GRCh38/hg38 human genome assembly): 21:36719822: TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or an mRNA molecule produced therefrom, or a cDNA molecule produced from the mRNA molecule.

For subjects that are genotyped or determined to be SIM2 reference, such subjects have a decreased risk of developing Parkinson's disease (compared to subjects that are heterozygous or homozygous for a SIM2 variant nucleic acid molecule). For subjects that are genotyped or determined to be heterozygous or homozygous for a SIM2 variant nucleic acid molecule, such subjects have an increased risk of developing Parkinson's disease and can be treated with a SIM2 agonist.

In any of the embodiments described herein, the subject in whom Parkinson's disease is prevented by administering a SIM2 agonist may be anyone at risk for developing Parkinson's disease including, but not limited to, subjects with a genetic predisposition for developing Parkinson's disease (e.g., genetic or family history). Additional risk factors for Parkinson's disease include, but are not limited to, age (average age of onset is about 60), gender (men are more likely to develop Parkinson's disease than women), environmental causes (such as, for example, exposure to farming chemicals, like pesticides and herbicides; Vietnam-era exposure to Agent Orange; and working with heavy metals, detergents and solvents), and head trauma (such as, for example, repeated blows to the head). In any of the embodiments described herein, the methods can be used to improve Parkinson's disease.

In any of the embodiments described herein, the SIM2 predicted loss-of-function polypeptide can be any SIM2 polypeptide having a partial loss-of-function, a complete loss-of-function, a predicted partial loss-of-function, or a predicted complete loss-of-function.

Any one or more (i.e., any combination) of the SIM2 variant nucleic acid molecules described herein can be used within any of the methods described herein to determine whether a subject has an increased or decreased risk of developing Parkinson's disease. The combinations of particular variants can form a mask used for statistical analysis of the particular correlation of SIM2 and an increased or decreased risk of developing Parkinson's disease. In some embodiments, the mask used for statistical analysis of the particular correlation of SIM2 and an increased or decreased risk of developing Parkinson's disease can exclude any one or more of these SIM2 variant nucleic acid molecules described herein.

In any of the embodiments described herein, the subject can have Parkinson's disease. In any of the embodiments described herein, the subject can be at risk of developing Parkinson's disease. In any of the embodiments described herein, the Parkinson's disease is idiopathic Parkinson's disease, vascular Parkinson's disease, or drug-induced Parkinson's disease. In some embodiments, the Parkinson's disease is idiopathic Parkinson's disease. In some embodiments, the Parkinson's disease is vascular Parkinson's disease. In some embodiments, the Parkinson's disease is drug-induced Parkinson's disease. In some embodiments, the Parkinson's disease is strict (ICD10-G20 definition). In some embodiments, the Parkinson's disease is broad (ICD10-G20 definition). In some embodiments, the Parkinson's disease is early-onset Parkinson's disease. In some embodiments, the Parkinson's disease is late-onset Parkinson's disease.

The present disclosure provides methods of treating a subject having Parkinson's disease or at risk of developing Parkinson's disease, the methods comprising administering a SIM2 agonist to the subject.

In any of the methods of treatment described herein, the subject being treated may comprise a SIM2 variant nucleic acid molecule. In some embodiments, the subject being treated is heterozygous for the SIM2 variant nucleic acid molecule. In some embodiments, the subject being treated is homozygous for the SIM2 variant nucleic acid molecule. The SIM2 variant nucleic acid molecule can be any of the SIM2 variant nucleic acid molecules disclosed herein. In some embodiments, the SIM2 variant nucleic acid molecule is a SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T: G, 21:36747923:TG:T, 21:36748041:GC:G, 21:36911797:G:A, 21:36876382:A:G, 21:36888743:C: A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

In some embodiments, the methods of treatment further comprise detecting the presence or absence of a SIM2 variant nucleic acid molecule in a biological sample from the subject. In some embodiments, the SIM2 variant nucleic acid molecule can be any of the SIM2 variant nucleic acid molecules disclosed herein. In some embodiments, the SIM2 variant nucleic acid molecule is a SIM2 variant genomic nucleic acid molecule that comprises the genetic variation 21:36719822:TG: T, 21:36726190:C: G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

In some embodiments, the methods of treatment further comprise detecting the presence or absence of any SIM2 variant nucleic acid molecule in a biological sample from the subject.

The present disclosure also provides methods of treating a subject with a Parkinson's disease therapeutic agent that treats or inhibits Parkinson's disease, wherein the subject has Parkinson's disease or is at risk of developing Parkinson's disease. The methods comprise determining whether the subject has a SIM2 variant nucleic acid molecule by obtaining or having obtained a biological sample from the subject, and performing or having performed a sequence analysis on the biological sample to determine if the subject has a genotype comprising the SIM2 variant nucleic acid molecule. In embodiments where the subject is SIM2 reference, the methods further comprise administering or continuing to administer the Parkinson's disease therapeutic agent in an amount that is the same as a standard dosage amount to the subject. In embodiments where the subject is heterozygous or homozygous for the SIM2 variant nucleic acid molecule, the methods further comprise administering or continuing to administer the Parkinson's disease therapeutic agent in an amount that is the same as or less than a standard dosage amount to the subject, and/or administering a SIM2 agonist to the subject. The presence of a SIM2 variant nucleic acid molecule indicates the subject has an increased risk of developing Parkinson's disease. In some embodiments, the subject is SIM2 reference. In some embodiments, the subject is heterozygous for a SIM2 variant nucleic acid molecule. In some embodiments, the subject is homozygous for a SIM2 variant nucleic acid molecule.

In any of the embodiments described herein, the SIM2 agonist is an example of a Parkinson's disease therapeutic agent. In some embodiments, the SIM2 variant nucleic acid molecule is a SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

For subjects that are genotyped or determined to be heterozygous or homozygous for a SIM2 variant nucleic acid molecule, such subjects can be administered a SIM2 agonist, as described herein.

Detecting the presence or absence of a SIM2 variant nucleic acid molecule in a biological sample from a subject and/or determining whether a subject has a SIM2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is SIM2 reference, the subject is administered a Parkinson's disease therapeutic agent in an amount that is the same as a standard dosage amount. In some embodiments, when the subject is heterozygous or homozygous for a SIM2 variant nucleic acid molecule, the subject is administered a Parkinson's disease therapeutic agent in an amount that is the same as or less than a standard dosage amount, and/or a SIM2 agonist.

In some embodiments, the treatment methods comprise detecting the presence or absence of a decrease in the expression of a SIM2 variant mRNA or polypeptide in a biological sample from the subject. In some embodiments, when the subject does not have a decrease in the expression of a SIM2 variant mRNA or polypeptide, the subject is administered a Parkinson's disease therapeutic agent in an amount that is the same as a standard dosage amount. In some embodiments, when the subject has a decrease in the expression of a SIM2 variant mRNA or polypeptide, the subject is administered a Parkinson's disease therapeutic agent in a standard dosage amount or less than a standard dosage amount, and/or a SIM2 agonist.

The present disclosure also provides methods of treating a subject with a Parkinson's disease therapeutic agent that treats or inhibits Parkinson's disease, wherein the subject has Parkinson's disease or is at risk of developing Parkinson's disease. The methods comprise determining whether the subject has a decrease in the expression of a SIM2 variant mRNA or polypeptide by obtaining or having obtained a biological sample from the subject, and performing or having performed an assay on the biological sample to determine if the subject has a decrease in the expression of a SIM2 variant mRNA or polypeptide. In embodiments where the subject does not have a decrease in the expression of a SIM2 variant mRNA or polypeptide, the methods further comprise administering or continuing to administer the Parkinson's disease therapeutic agent in an amount that is the same as a standard dosage amount. In embodiments where the subject has a decrease in the expression of a SIM2 variant mRNA or polypeptide, the methods further comprise administering or continuing to administer the Parkinson's disease therapeutic agent in an amount that is the same as or less than a standard dosage amount to the subject, and/or administering a SIM2 agonist to the subject. The presence of a decrease in the expression of a SIM2 variant mRNA or polypeptide indicates the subject has an increased risk of developing Parkinson's disease. In some embodiments, the subject has a decrease in the expression of a SIM2 variant mRNA or polypeptide. In some embodiments, the subject does not have a decrease in the expression of a SIM2 variant mRNA or polypeptide. In any of the embodiments described herein, the SIM2 agonist is an example of a Parkinson's disease therapeutic agent. In some embodiments, the SIM2 variant nucleic acid molecule is a SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

Detecting a decrease in the expression of a SIM2 variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In some embodiments, the treatment methods comprise detecting the presence or absence of a SIM2 variant polypeptide in a biological sample from the subject. In some embodiments, when the subject does not have a SIM2 variant polypeptide, the subject is administered a Parkinson's disease therapeutic agent in an amount that is the same as a standard dosage amount. In some embodiments, when the subject has a SIM2 variant polypeptide, the subject is administered a Parkinson's disease therapeutic agent in an amount that is the same as or less than a standard dosage amount, and/or is administered a SIM2 agonist.

The present disclosure also provides methods of treating a subject with a Parkinson's disease therapeutic agent that treats or inhibits Parkinson's disease, wherein the subject has Parkinson's disease or is at risk of developing Parkinson's disease. The methods comprise determining whether the subject has a SIM2 variant polypeptide by obtaining or having obtained a biological sample from the subject and performing or having performed an assay on the biological sample to determine if the subject has a SIM2 variant polypeptide. When the subject does not have a SIM2 variant polypeptide, the subject is administered the Parkinson's disease therapeutic agent in an amount that is the same as a standard dosage amount. When the subject has a SIM2 variant polypeptide, the subject is administered the Parkinson's disease therapeutic agent in an amount that is the same as or less than a standard dosage amount, and/or is administered a SIM2 agonist. The presence of a SIM2 variant polypeptide indicates the subject has an increased risk of developing Parkinson's disease. In some embodiments, the subject has a SIM2 variant polypeptide. In some embodiments, the subject does not have a SIM2 variant polypeptide.

The present disclosure also provides methods of preventing a subject from developing Parkinson's disease by administering a Parkinson's disease therapeutic agent that prevents Parkinson's disease. In some embodiments, the methods comprise determining whether the subject has a SIM2 variant polypeptide by obtaining or having obtained a biological sample from the subject and performing or having performed an assay on the biological sample to determine if the subject has a SIM2 variant polypeptide. When the subject does not have a SIM2 variant polypeptide, the subject is administered the Parkinson's disease therapeutic agent in an amount that is the same as a standard dosage amount. When the subject has a SIM2 variant polypeptide, the subject is administered the Parkinson's disease therapeutic agent in an amount that is the same as or less than a standard dosage amount, and/or is administered a SIM2 agonist. The presence of a SIM2 variant polypeptide indicates the subject has an increased risk of developing Parkinson's disease. In some embodiments, the subject has a SIM2 variant polypeptide. In some embodiments, the subject does not have a SIM2 variant polypeptide.

In some embodiments, a subject having a SIM2 variant nucleic acid molecule as described herein can undergo gene editing in order to remove and replace the SIM2 variant nucleic acid molecule. In some embodiments, the methods of gene editing include, but are not limited to programmable nucleases (gene editing tools) such as zinc finger nucleases (ZFNs), transcription activator-like effector nucleases (TALENs), clustered regularly interspaced short palindromic repeats (CRISPR/Cas), base editing (BE; cytosine BE (CBE)/adenine BE (ABE)), and prime editing (PE) (see, Phan et al., Genes, 2023, 14, 1-21, which is incorporated herein by reference in its entirety). Suitable delivery systems include, but are not limited to, virus (AAV and lentivirus), and non-virus (silica nanoconstruct (SN), engineered virus like particle (eVLP), nanoparticle).

In some embodiments, the gene editing methods comprise a nuclease agent that induces one or more nicks or double-strand breaks at a recognition sequence(s) or a DNA-binding protein that binds to a recognition sequence within a SIM2 genomic nucleic acid molecule. The recognition sequence can be located within a coding region of the SIM2 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the SIM2 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a SIM2 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of SIM2 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a SIM2 genomic nucleic acid molecule or it can be a nickase that creates a single-strand break in a SIM2 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of SIM2 genomic nucleic acid molecules can be generated by contacting a cell with a Cas protein and one or more gRNAs that hybridize to one or more gRNA recognition sequences within a target genomic locus in the SIM2 genomic nucleic acid molecule. The gRNA recognition sequence can include or be proximate to the start codon of a SIM2 genomic nucleic acid molecule or the stop codon of a SIM2 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a SIM2 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-31, where Nis any DNA nucleotide and is immediately 3′ of the gRNA recognition sequence of the non-complementary strand of the target DNA. As such, the PAM sequence of the complementary strand would be 5′-CCN-31, where N is any DNA nucleotide and is immediately 5′ of the gRNA recognition sequence of the complementary strand of the target DNA.

A gRNA is an RNA molecule that binds to a Cas protein and targets the Cas protein to a specific location within a SIM2 genomic nucleic acid molecule. An exemplary gRNA is a gRNA effective to direct a Cas enzyme to bind to or cleave a SIM2 genomic nucleic acid molecule, wherein the gRNA comprises a DNA-targeting segment that hybridizes to a gRNA recognition sequence within the SIM2 genomic nucleic acid molecule. Exemplary gRNAs comprise a DNA-targeting segment that hybridizes to a gRNA recognition sequence present within a SIM2 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the SIM2 genomic nucleic acid molecule. The Cas protein can cleave the nucleic acid molecule at a site within or outside of the nucleic acid sequence present in the SIM2 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the SIM2 genomic nucleic acid molecule to which a DNA-targeting segment of a gRNA will bind.

Such methods can result, for example, in a SIM2 genomic nucleic acid molecule in which a region of the SIM2 genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the SIM2 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

Detecting the presence or absence of a SIM2 variant polypeptide in a biological sample from a subject and/or determining whether a subject has a SIM2 variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the SIM2 agonist can be a stimulator such that the expression of SIM2 mRNA is increased. In some embodiments, the SIM2 agonist can be a stimulator such that the expression of SIM2 polypeptide is increased. In some embodiments, the SIM2 agonist can be a stimulator such that the activity of SIM2polypeptide is increased.

In some embodiments, the SIM2 agonist is a small molecule. In some embodiments, the small molecule is low molecular weight (<900 daltons) organic compound.

In some embodiments, the SIM2 agonist comprises wild type SIM2 protein or a Cox-2 inhibitor (e.g., celecoxib, rofecoxib, etoricoxib, valdecoxib, and parecoxib).

In some embodiments, the Parkinson's disease therapeutic agents include, but are not limited to, carbidopa, levodopa, a combination of carbidopa and levodopa, droxidopa, melevodopa, foscarbidopa, foslevodopa, a dopamine agonist (such as, for example, pramipexole, rotigotine, apomorphine, bromocriptine, pergolide, ropinirole, piribedil, cabergoline, and lisuride), a monoamine oxidase B (MAO B) inhibitor (such as, for example, selegiline, rasagiline, and safinamide), a catechol O-methyltransferase (COMT) inhibitor (such as, for example, entacapone, tolcapone, and opicapone), an anticholinergic (such as, for example, benztropine and trihexyphenidyl), amantadine, an adenosine receptor antagonist (A2A receptor antagonists) (such as, for example, istradefylline), pimavanserin, benserazide, biperiden, bornaprine, quetiapine, rivastigmine, diphenhydramine, and hyoscyamine. In some embodiments, the Parkinson's disease therapeutic agent can be combined with a SIM2 agonist. The Parkinson's disease therapeutic agents may be delayed or avoided altogether by treatment with a SIM2 agonist, as described herein.

In some embodiments, the dose of the Parkinson's disease therapeutic agents that treat, prevent, or inhibit Parkinson's disease can be decreased by about 10%, by about 20%, by about 30%, by about 40%, by about 50%, by about 60%, by about 70%, by about 80%, or by about 90% for subjects that are heterozygous or homozygous for a SIM2 variant nucleic acid molecule (i.e., a less than the standard dosage amount) compared to subjects that are SIM2 reference (who may receive a standard dosage amount). In some embodiments, the dose of the Parkinson's disease therapeutic agents that treat, prevent, or inhibit Parkinson's disease can be decreased by about 10%, by about 20%, by about 30%, by about 40%, or by about 50%. In some embodiments, the dose of the Parkinson's disease therapeutic agents that treat, prevent, or inhibit Parkinson's disease can be decreased by about 10%, by about 20%, by about 30%, by about 40%, by about 50%, by about 60%, by about 70%, by about 80%, or by about 90% for subjects that are homozygous for a SIM2 variant nucleic acid molecule compared to subjects that are heterozygous for a SIM2 variant nucleic acid molecule. In addition, subjects that are heterozygous or homozygous for a SIM2 variant nucleic acid molecule can be administered the Parkinson's disease therapeutic agents less frequently compared to subjects that are SIM2 reference.

Administration of the Parkinson's disease therapeutic agents that treat, prevent, or inhibit Parkinson's disease and/or SIM2 agonists can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the Parkinson's disease therapeutic agents and/or SIM2 agonists can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in Parkinson's disease, a decrease/reduction in the severity of Parkinson's disease (such as, for example, a reduction or inhibition of development of Parkinson's disease), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to Parkinson's disease, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected subject, following administration of the agent or composition comprising the agent. A prophylactic effect may comprise a complete or partial avoidance/inhibition or a delay of Parkinson's disease development/progression (such as, for example, a complete or partial avoidance/inhibition or a delay), and an increased survival time of the affected subject, following administration of a therapeutic protocol. Treatment of Parkinson's disease encompasses the treatment of a subject already diagnosed as having any form of Parkinson's disease at any clinical stage or manifestation, the delay of the onset or evolution or aggravation or deterioration of the symptoms or signs of Parkinson's disease, and/or preventing and/or reducing the severity of Parkinson's disease.

In some embodiments, the SIM2 agonist and the Parkinson's disease therapeutic agent are disposed within a pharmaceutical composition. In some embodiments, the SIM2agonist is disposed within a first pharmaceutical composition and the Parkinson's disease therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

In some embodiments, the subject is also administered Vitamin B5. In some embodiments, Vitamin B5 is administered prior to administration of the SIM2 agonist. In some embodiments, Vitamin B5 is administered after administration of the SIM2 agonist. In some embodiments, Vitamin B5 is administered concurrently with the administration of the SIM2 agonist.

The present disclosure also provides methods of identifying a subject having an increased risk of developing Parkinson's disease. In some embodiments, the method comprises determining or having determined in a biological sample obtained from the subject the presence or absence of a SIM2 variant nucleic acid molecule (such as a genomic nucleic acid molecule, mRNA molecule, and/or cDNA molecule). When the subject lacks a SIM2 variant nucleic acid molecule (i.e., the subject is genotypically categorized as SIM2 reference), then the subject has a decreased risk of developing Parkinson's disease (compared to subjects that are heterozygous or homozygous for a SIM2 variant nucleic acid molecule). When the subject has a SIM2 variant nucleic acid molecule (i.e., the subject is heterozygous or homozygous for a SIM2 variant nucleic acid molecule), then the subject has an increased risk of developing Parkinson's disease. In some embodiments, the SIM2 variant nucleic acid molecule is a SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

Having two copies of a SIM2 variant nucleic acid molecule may result in a greater risk of a subject for developing Parkinson's disease than having a single copy of a SIM2 variant nucleic acid molecule. Without intending to be limited to any particular theory or mechanism of action, it is believed that a single copy of a SIM2 variant nucleic acid molecule (i.e., heterozygous for a SIM2 variant nucleic acid molecule) increases the risk of a subject for developing Parkinson's disease and it is also believed that having two copies of a SIM2 variant nucleic acid molecule (i.e., homozygous for a SIM2 variant nucleic acid molecule) may increase the risk even more of a subject for developing Parkinson's disease, relative to a subject with a single copy.

Determining whether a subject has a SIM2 variant nucleic acid molecule in a biological sample from a subject and/or determining whether a subject has a SIM2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing Parkinson's disease, the subject is administered a Parkinson's disease therapeutic agent, and/or a SIM2 agonist, as described herein. For example, when the subject is SIM2 reference, and therefore has a decreased risk of developing Parkinson's disease, the subject is administered a Parkinson's disease therapeutic agent in an amount that is the same as a standard dosage amount. In some embodiments, when the subject is heterozygous or homozygous for a SIM2 variant nucleic acid molecule, the subject is administered the Parkinson's disease therapeutic agent in an amount that is the same as or less than a standard dosage amount, and/or is administered a SIM2 agonist. In some embodiments, the subject is SIM2 reference. In some embodiments, the subject is heterozygous for a SIM2 variant nucleic acid molecule. In some embodiments, the subject is homozygous for a SIM2 variant nucleic acid molecule.

The present disclosure also provides methods of determining a subject's aggregate burden, or risk score, of having two or more SIM2 variant nucleic acid molecules, and/or two or more SIM2 variant polypeptides associated with an increased risk of developing Parkinson's disease. The aggregate burden is the sum of two or more genetic variants that can be carried out in an association analysis with Parkinson's disease. In some embodiments, the subject is homozygous for one or more SIM2 variant nucleic acid molecules associated with an increased risk of developing Parkinson's disease. In some embodiments, the subject is heterozygous for one or more SIM2 variant nucleic acid molecules associated with an increased risk of developing Parkinson's disease. When the subject has a lower aggregate burden, the subject has a decreased risk of developing Parkinson's disease, and the subject is administered or continued to be administered the Parkinson's disease therapeutic agent in an amount that is the same as a standard dosage amount. When the subject has a higher aggregate burden, the subject has an increased risk of developing Parkinson's disease and the subject is administered or continued to be administered the Parkinson's disease therapeutic agent in an amount that is the same as or less than a standard dosage amount, and/or a SIM2 agonist. The higher the aggregate burden, the higher the risk of developing Parkinson's disease.

In some embodiments, the aggregate burden may be divided into quintiles, e.g., top quintile, second quintile, intermediate quintile, fourth quintile, and bottom quintile, wherein the top quintile of aggregate burden corresponds to the highest risk group and the bottom quintile of aggregate burden corresponds to the lowest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with Parkinson's disease in the top 10%, top 20%, top 30%, top 40%, or top 50% of p-value range for the association. In some embodiments, each of the identified genetic variants comprise the genetic variants having association with Parkinson's disease with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with Parkinson's disease with p-value of less than 5×10⁻⁸. In some embodiments, the identified genetic variants comprise genetic variants having association with Parkinson's disease in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0,from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the top decile, quintile, or tertile in a reference population. The threshold of the aggregate burden can be determined on the basis of the nature of the intended practical application and the risk difference that would be considered meaningful for that practical application.

In embodiments where the aggregate burden is determined for SIM2 genetic variants associated with Parkinson's disease, then the aggregate burden represents a subject's risk score for developing Parkinson's disease. In some embodiments, the aggregate burden or risk score includes the SIM2 variant genomic nucleic acid molecule that comprises the genetic variation 21:36719822:TG:T, 21:36726190:C:G, 21:36731044:G C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule. In some embodiments, a subject's aggregate burden can be determined for SIM2 genetic variants associated with Parkinson's disease in combination with additional genetic variants for other genes also associated with Parkinson's disease to produce a polygenic risk score (PRS) for developing Parkinson's disease. In some embodiments, the PRS includes the SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

The present disclosure also provides methods of detecting the presence or absence of a SIM2 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any SIM2 variant nucleic acid molecule, preliminary processing designed to isolate or enrich the biological sample for the genomic DNA can be employed. A variety of techniques may be used for this purpose. When detecting the level of any SIM2 variant nucleic acid molecule, different techniques can be used to enrich the biological sample with mRNA molecules. Various methods to detect the presence or level of an mRNA molecule or the presence of a particular variant genomic DNA locus can be used.

In some embodiments, detecting a SIM2 variant nucleic acid molecule in a subject comprises performing a sequence analysis on a biological sample obtained from the subject to determine whether a SIM2 genomic nucleic acid molecule in the biological sample, and/or a SIM2 mRNA molecule in the biological sample, and/or a SIM2 cDNA moleculeproduced from an mRNA molecule in the biological sample, is present in the sample. In some embodiments, the methods detect the SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or an mRNA molecule produced therefrom, or a cDNA molecule produced from the mRNA molecule.

In some embodiments, the methods of detecting the presence or absence of a SIM2 variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a SIM2 genomic nucleic acid molecule or mRNA molecule, and if mRNA, optionally reverse transcribing the mRNA into cDNA. Such assays can comprise, for example determining the identity of these positions of the particular SIM2 nucleic acid molecule. In some embodiments, the method is an in vitro method.

In some embodiments, the determining step, detecting step, or sequence analysis comprises sequencing at least a portion of the nucleotide sequence of the SIM2 genomic nucleic acid molecule, the SIM2 mRNA molecule, or the SIM2 cDNA molecule in the biological sample that comprises a genetic variation compared to the corresponding SIM2 reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only a SIM2 genomic nucleic acid molecule is analyzed. In some embodiments, only a SIM2 mRNA is analyzed. In some embodiments, only a SIM2 cDNA obtained from the SIM2 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a SIM2 variant genomic sequence, variant mRNA sequence, or variant cDNA sequence and not the corresponding SIM2 reference sequence under stringent conditions and determining whether hybridization has occurred.

In some embodiments, the determining step, detecting step, or sequence analysis comprises: a) amplifying at least a portion of the SIM2 nucleic acid molecule that encodes the SIM2 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a SIM2 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2× SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to SIM2 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to SIM2 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the SIM2 variant nucleic acid molecules disclosed herein. The primers described herein can be used to amplify any SIM2 variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding a SIM2 reference genomic nucleic acid molecule, a SIM2 reference mRNA molecule, and/or a SIM2 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, agonists, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides Parkinson's disease therapeutic agents that treat, prevent, or inhibit Parkinson's disease for use in the treatment or prevention of Parkinson's disease in a subject having a SIM2 variant nucleic acid molecule. Any of the Parkinson's disease therapeutic agents that treat, prevent, or inhibit Parkinson's disease described herein can be used herein. Any of the SIM2 variant nucleic acid molecules disclosed herein can be used herein. In some embodiments, the SIM2 variant nucleic acid molecule is a SIM2 variant genomic nucleic acid molecule that comprises the genetic variation 21:36719822:TG: T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C: A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

The present disclosure also provides uses of Parkinson's disease therapeutic agents that treat, prevent, or inhibit Parkinson's disease for use in the preparation of a medicament for treating or preventing Parkinson's disease in a subject having a SIM2 variant nucleic acid molecule. Any of the Parkinson's disease therapeutic agents that treat, prevent, or inhibit Parkinson's disease described herein can be used herein. Any of the SIM2 variant nucleic acid molecules disclosed herein can be used herein. In some embodiments, the SIM2 variant nucleic acid molecule is a SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

The present disclosure also provides SIM2 agonists for use in the treatment or prevention of Parkinson's disease in a subject that is heterozygous or homozygous for a SIM2 variant nucleic acid molecule. Any of the SIM2 agonists disclosed herein can be used herein. Any of the SIM2 variant nucleic acid molecules disclosed herein can be used herein.

In some embodiments, the SIM2 variant nucleic acid molecule is a SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

The present disclosure also provides SIM2 agonists in the preparation of a medicament for treating or preventing Parkinson's disease in a subject that is heterozygous or homozygous for a SIM2 variant nucleic acid molecule. Any of the SIM2 agonists disclosed herein can be used herein. Any of the SIM2 variant nucleic acid molecules disclosed herein can be used herein. In some embodiments, the SIM2 variant nucleic acid molecule is a SIM2 variant genomic nucleic acid molecule that comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C, or is an mRNA molecule produced therefrom, or is a cDNA molecule produced from the mRNA molecule.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1

### General Methods

Participating cohorts

Genome-wide association analyses were performed in seven cohorts including 470,000 samples from the U.K. Biobank cohort and 175,000 from the Geisinger Health System MyCode cohort. Other datasets include: over 40,000 participants from the University of Pennsylvania Penn Medicine BioBank, ˜30,000 participants from the Mount Sinai BioMe BioBank, 49,000 from the Colorado Center for Personalized Medicine Biobank, and 40,000 from the UCLA ATLAS Community Health Initiative. We also included >115,000 participants from the MAYO-RGC Project Generation. This study was reviewed and approved by the Mayo Clinic IRB (#09-007763).

Phenotype definitions

Parkinson's disease cases were adjudicated in each cohort on the basis of one or more of the following criteria: 1) an electronic health record history of Parkinson's disease (using International Classification of Diseases, Tenth Revision (ICD-10) diagnosis codes G20, in at least one inpatient encounter or at least two outpatient encounters or if noted as a cause of death; 2) for broad parkinson's disease definition-—an electronic health record with ICD-10 code for Lewy body demetia (G31.83) or dementia in other diseases including parkinson's disease (F02.80) or self-reported Parkinson's disease; 3) also for broad parkinson's disease definition—a reported family history of disease for either mother, father, or siblings.

### Exome Sequencing and Whole-Genome Genotyping

For analyses of common variants, array genotyping data and imputation were performed with the use of the TOPMed reference panel. Exome sequencing was performed at the Regeneron Genetics Center using a custom automated sample preparation approach. Samples were captured with IDT xGen v1 or Twist Comprehensive Exome probes and sequenced using Illumina HiSeq 2500-v4 or Illumina NovaSeq instruments, with 75-bp paired-end reads and two index reads. The GRCh38 human genome reference sequence and Ensembl, version 85, gene definitions were used for variant identification and annotation. For the COLORADO, MAYO-CLINIC and UCLA cohorts sequenced with Twist, probes also included the Twist Diversity SNP panel, for which multi-point refinement was conducted using GLIMPSE prior to further genotype QC and imputation. For exome coding variants, variants are classified from most to least deleterious in the following order: frameshift, stop-gain, stop-loss, splice acceptor, splice donor, in-frame insertion or deletion (indel), missense, and other annotations. Frameshift, stop-gain, stop-loss, splice-acceptor, and splice-donor alleles were categorized as predicted loss-of-function variants. Missense variants were classified using computer modeling to predict functional effects with five algorithms: SIFT53, Polyphen-2 HDIV54, Polyphen-2 HVAR54, LRT55 and MutationTaster56.To account for the fact that different genes have different types and frequencies of potentially causative variants, the functional annotation of the variants in each gene was used to generate seven pseudo-genotypes based on the combined variant burden: predicted loss-of-function variants; predicted loss-of-function variants plus missense variants that were predicted to be deleterious by five of five algorithms; predicted loss-of-function variants plus missense variants that were predicted to be deleterious by at least one of five algorithms; predicted loss-of-function variants plus any missense variants; missense variants that were predicted to be deleterious by five of five algorithms; missense variants that were predicted to be deleterious by at least one of five algorithms; and finally, any missense variants at all (these categories are similar to those used previously). The alternative allele frequency and functional annotation of each variant are used to generate seven genotypes based on the combined variant burden: pLoF variants with an alternative-allele frequency thresholds of 1%, 0.1%, 0.01% and singletons, pLoF variants plus missense variants that were predicted to be deleterious and had an alternative-allele frequency thresholds of 1%, 0.1%, 0.01% and singletons.

### Statistical Analysis

Associations between genotypes and phenotypes were estimated by fitting linear regression models (for quantitative traits) or Firth bias-corrected logistic regression models (for binary traits) using the REGENIE software, version 2+58. Analyses were stratified according to cohort and ancestry and were adjusted for age, age squared, sex, age-by-sex, and age squared-by-sex interaction terms; experimental batch-related covariates; the first 10 common variant-derived genetic principal components; the first 20 rare variant-derived principal components; and a polygenic score generated by REGENIE, which robustly adjusts for relatedness and population structure. A meta-analysis of association results across cohorts and ancestries with a fixed-effect inverse-variance-weighted approach was performed. The results for TOPMED imputed data for common variants are defined by minor allele frequency greater than 0.5%, and exome sequenced rare coding variants are defined by that had a minor allele count greater than five in both cases and controls. For gene burden analyses, each of the variant-burden categories mentioned above was tested at four thresholds of alternate-allele frequencies: alternative-allele frequencies of less than 1%; alternative-allele frequencies of less than 0.5%; alternative-allele frequencies of less than 0.1%; and alternative-allele frequencies of less than 0.01%. These seven categories and four thresholds produce 28 pseudo-genotypes for each gene, but they are not fully independent of one another, given the overlapping annotations and frequency thresholds. Thus, an appropriate adjusted Bonferroni significance level for these variant-burden tests was calculated, using a method recommended by a review of multiple-testing correction methods in non-independent genetic tests. Calculating the effective number of independent tests based on the correlation matrix of these variant-burden tests in our meta-analysis resulted in a value of 9.002158 tests per gene, which, when multiplied by the number of genes tested (19,446) and used as a correction factor for an alpha level of 0.05, resulted in an exome-wide level of significance at a P value of 2.86e-07.

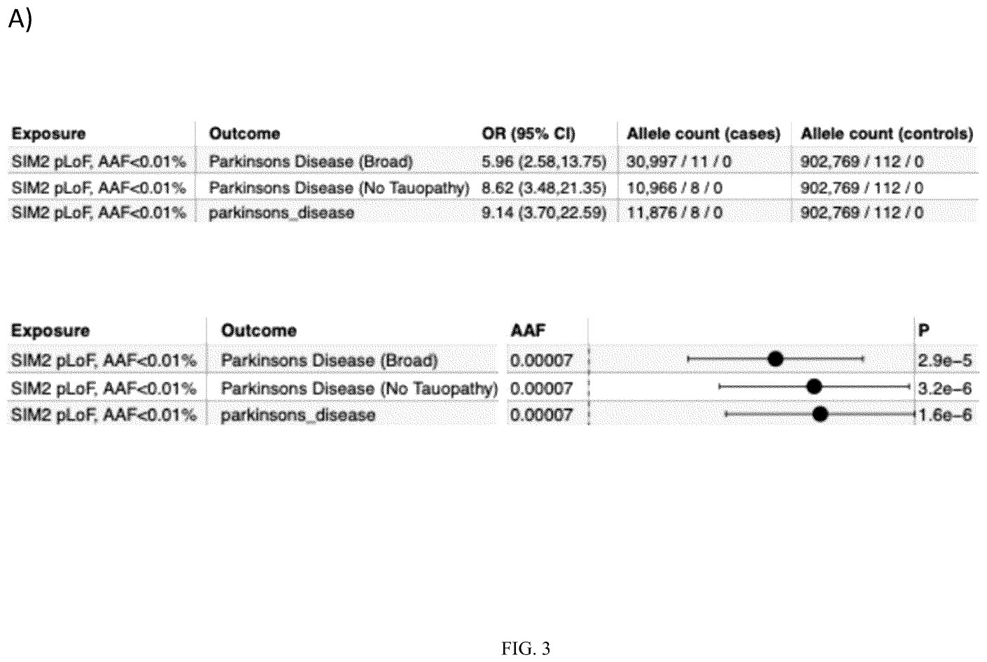

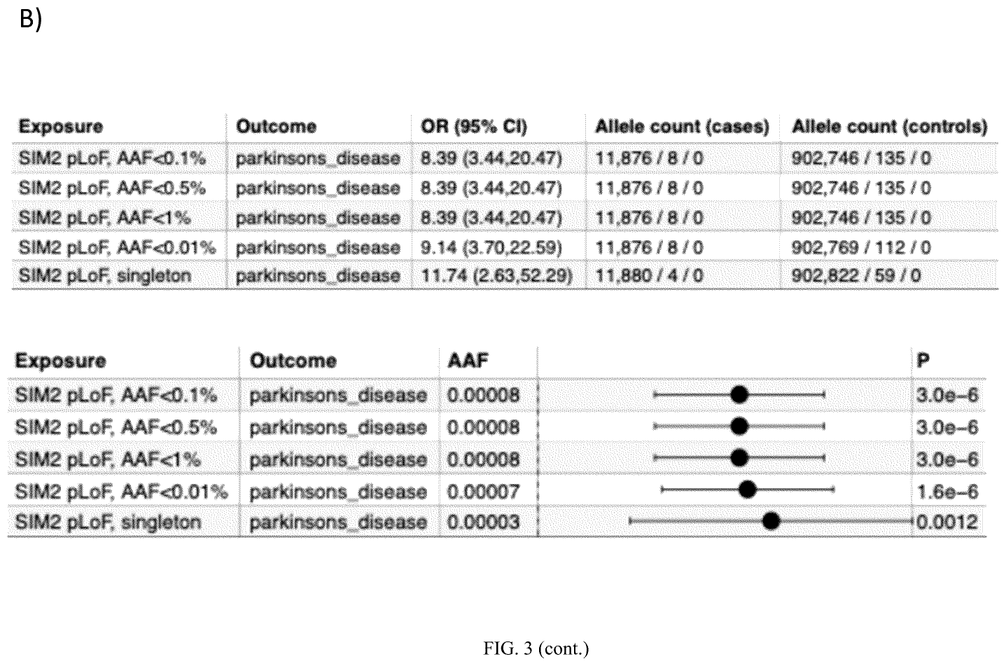

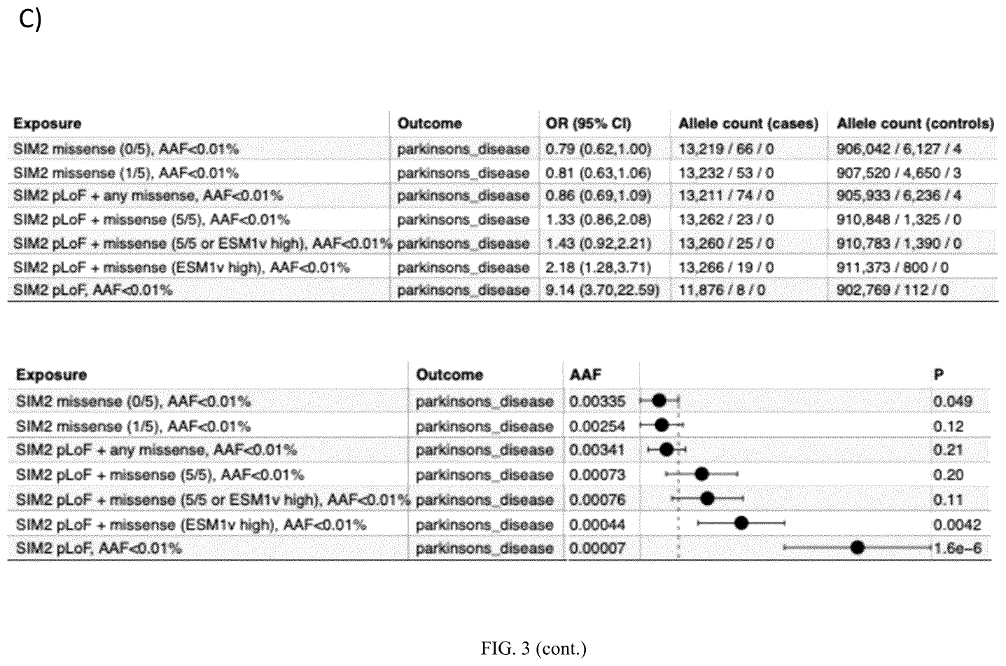

FIG. 3 (Panels A, B, and C) shows a rare pLoF mask and increasing risk of PD; risk arising from loss of SIM2 is maintained in broad and strict Parkinson's disease definitions (Panel A); more common pLoF masks maintain risk with same case carriers (Panel B); and missense only masks (top) do not show risk—pointing to a clear loss of function mechanism (Panel C).

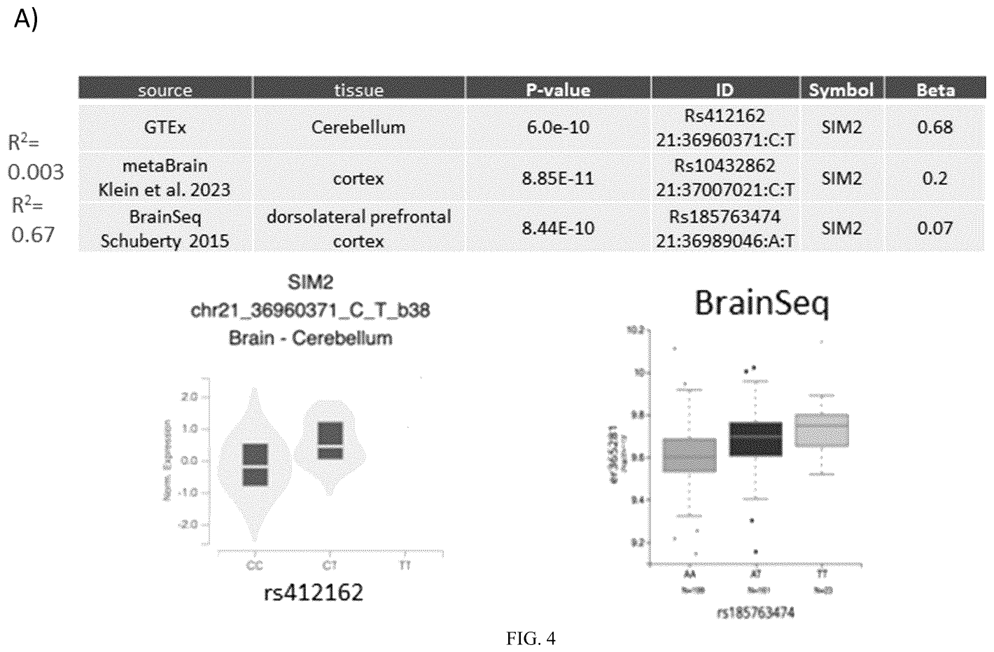

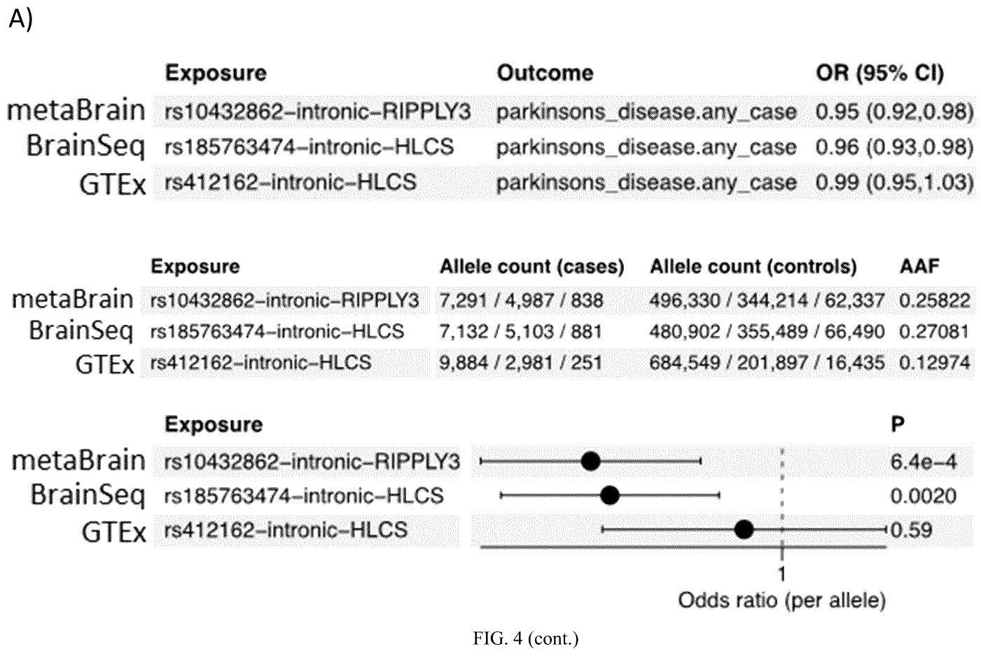

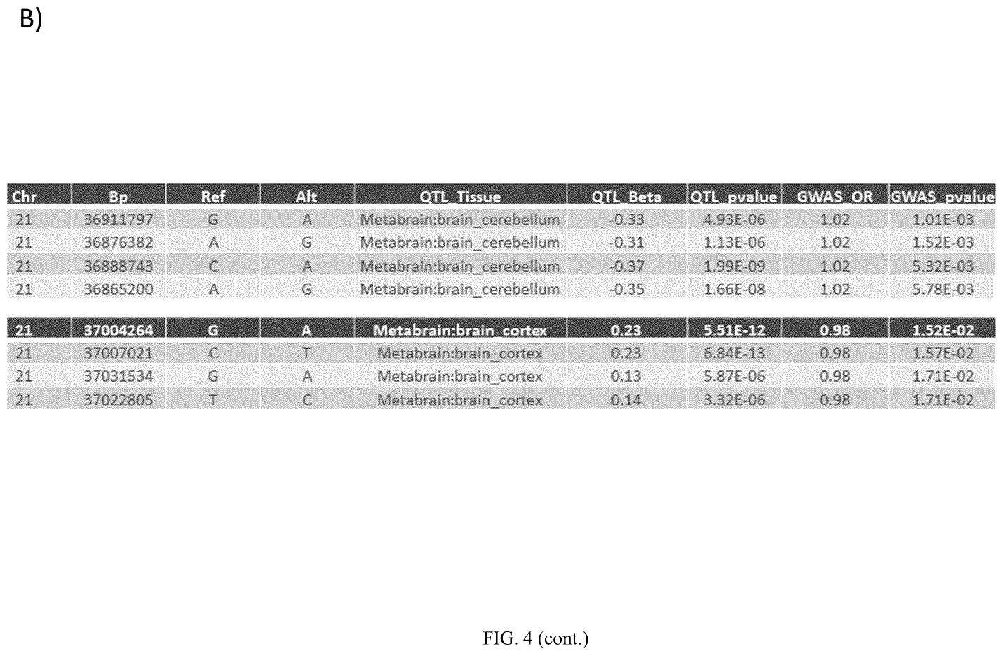

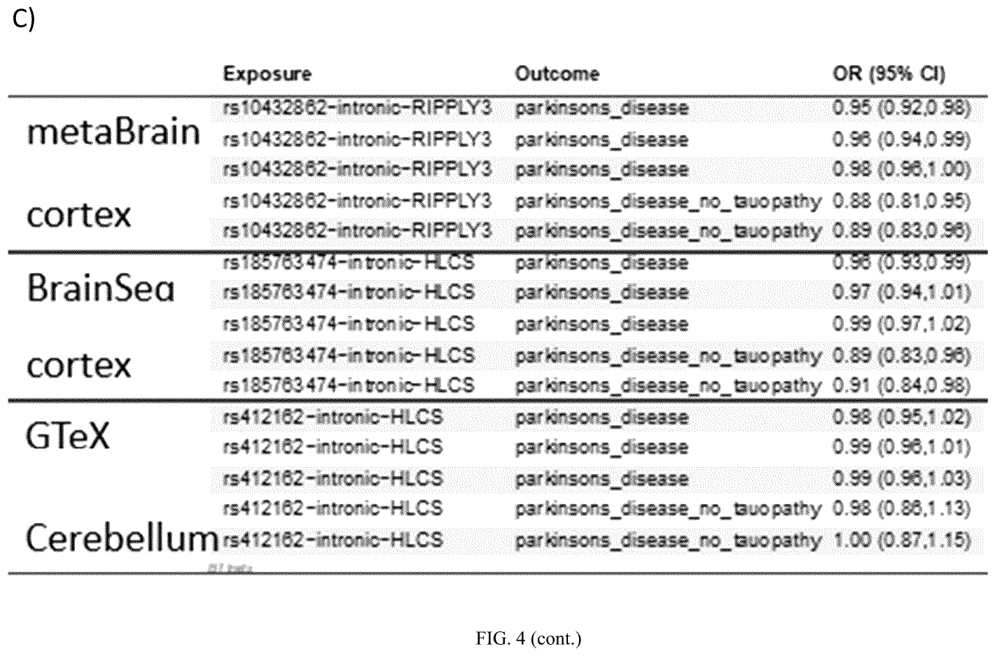

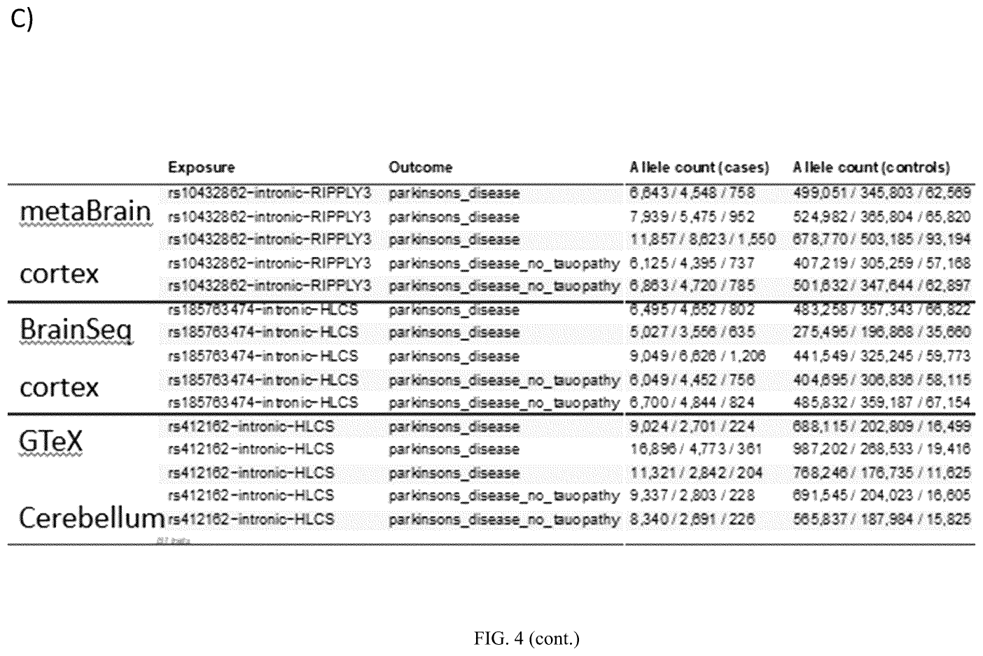

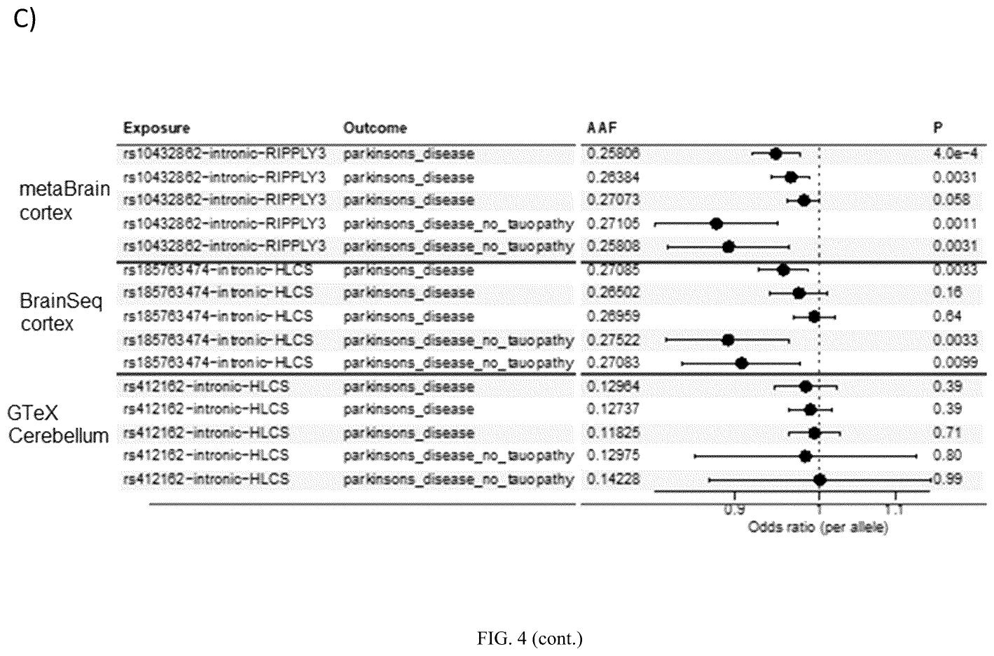

FIG. 4 (Panels A, B, and C) shows eQTLs increasing SIM2 show protection from PD; top eQTLs increasing SIM2 expression from brain tissues (Panel A); Metabrain eQTLs are consistent-decreasing SIM2 shows nominal risk (cerebellum), increasing SIM2 shows protection (cortex) (Panel B); and eQTLs increasing SIM2 shows nominal protection from PD (Panel C).

The data presented herein fits the hypothesis that SIM2 recruits PRKN to mitochondria to promote mitophagy, and its LoF prevents mitophagy and causes dopaminergic neuronal cell death (similar to loss of PINK1 or PRKN itself). The additional eQTL data is supporting the same direction—eQTLs decreasing expression in brain tissues (Cortex and cerebellum) are showing nominal risk, and eQTLs increasing expression are showing nominal protection.

### Example 2

### SIM2 Expression in the Context of Neurological Disease

To assess SIM2 expression in the context of neurological disease, SIM2 protein expression was determined in various cell types and from mouse and human brain tissues. SIM2 protein was expressed in human HepG2 liver cancer cell line as well as in SHSY5Y human neuroblastoma cell line. SIM2 expression in neuron was further validated in primary mouse cortical neuron. In addition to SIM2 expression from in vitro cultures, SIM2 was also expressed in human brain and mouse brain tissues, specifically in ventral midbrain region that is particularly affected in Parkinson's disease.

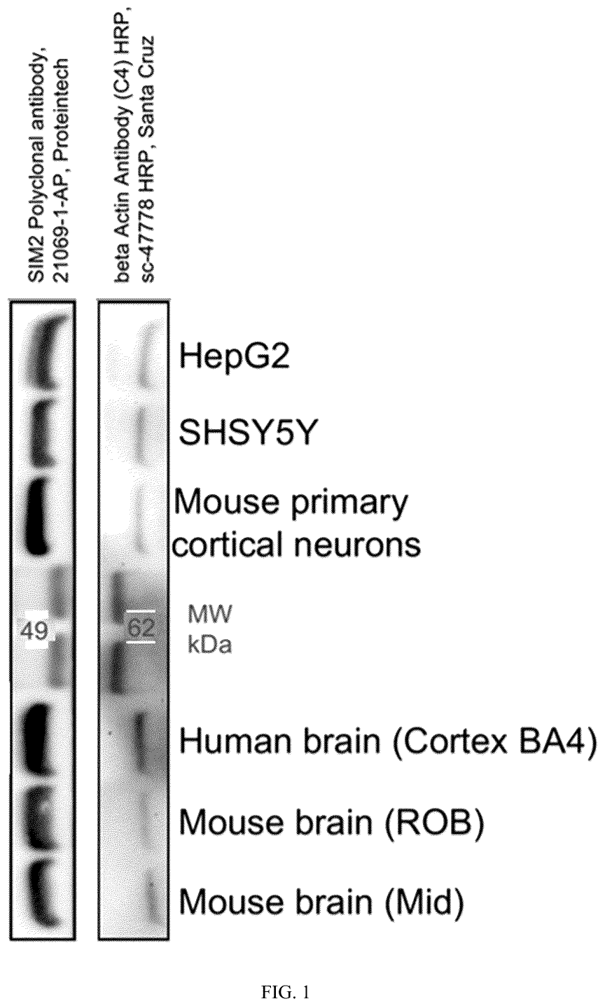

HepG2, SHSY5Y and mouse primary neuron cultures were collected and prepared with lysis buffer (50 mM Tris-HCl, 150 mM NaCl, 1% Triton X-100, and 5 mM EDTA (Thermo Scientific; J62289.AP) and protease and phosphatase inhibitors (Cell signaling; 5872). Cell lysates were placed on ice for 20 minutes and centrifuged at 13,000 rpm for 20 minutes to collect the supernatant. For human and mouse brain tissues, RIPA buffer (Thermo Scientific; 89900) and 3 mm tungsten carbide balls (Qiagen; NC9740573) were added and the resulting sample was lysed using tissuelyser LT (Qiagen; 85600) for 15 minutes at 50 Hz. The sample was further centrifuged at 13,000 rpm for 20 minutes to collect the supernatant. Lysates (20 μg for cell lysates and 50 ug for tissues) were separated with 4-12% Bis-Tris NuPAGE gel (Thermo Fisher Scientific) and blotted with the following primary antibodies: SIM2 (1:1000; Proteintech; 21069-1-AP) and beta actin (1:5000; Santa Cruz; sc-47778 HRP). HRP-conjugated secondary antibodies (1:3000; Bio Rad; 1706515) and SuperSignal West Dura (Thermo Scientific; 34075) for SIM2 and Pierce ECL western blotting substrates (Thermo Scientific; 32106) for beta actin were used for chemiluminescent detection. For TaqMan™ analysis, RNA was extracted from HEK293T cells using TRIzol (Invitrogen; 15596026) according to the manufacturer's protocol. Probe information for SIM2 and beta actin are: SIM2 probe sequence (TGCAGTGGCTACTTGAAGATCAGGC; SEQ ID NO: 1); SIM2 forward primer (TGCAGCGGATACAAGGTCATC; SEQ ID NO: 2); SIM2 reverse primer (GCAGGAGTCG TACAGGGACAT; SEQ ID NO: 3); beta actin probe sequence (CAAGATCATTGCTCCTCCTGAG CGC; SEQ ID NO: 4); beta actin forward primer (GGCACCCAGCACAATGAAG; SEQ ID NO: 5); beta actin reverse primer (GCCGATCCACACGGAGTA; SEQ ID NO: 6). Results are depicted in FIG. 1.

### Example 3

Knockdown of SIM2 Protein and mRNA by shRNA Validates Specificity of the Antibody

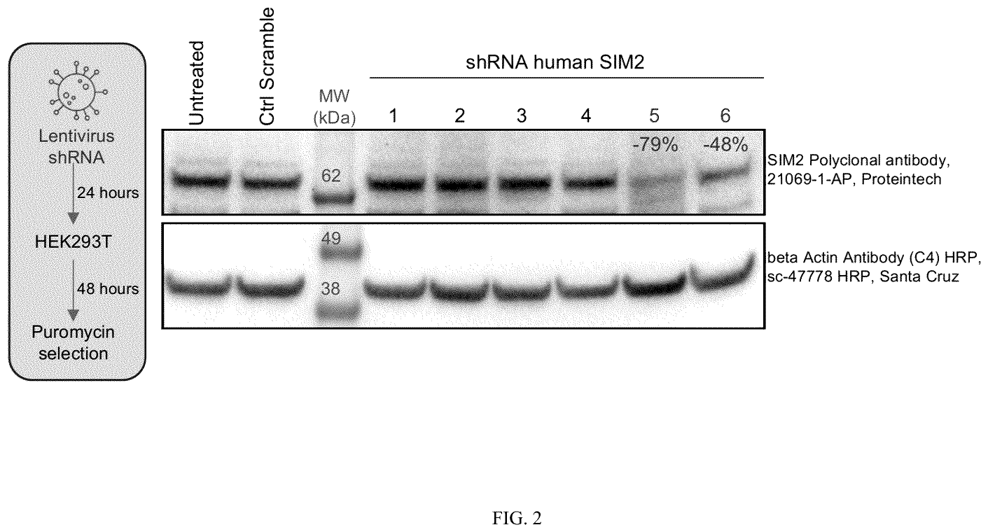

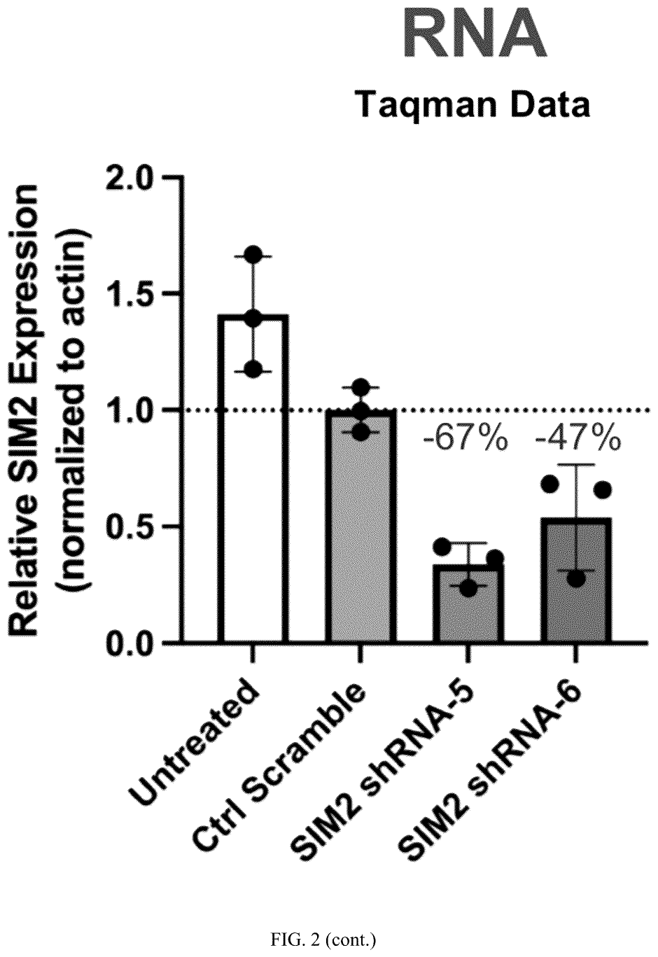

To validate the SIM2 antibodies used to assess SIM2 expression, the lentivirus was generated to express either scrambled control or shRNA against SIM2. Two of the shRNA against SIM2 led to robust knockdown of SIM2 mRNA, which correlated with SIM2 protein knockdown. HEK293T cells were transduced with the lentivirus to express scrambled control shRNA or SIM2 shRNAs (sequences 1 through 6). Cells were selected at the presence of 2 ug/mL puromycin. SIM2 protein level and SIM2 RNA level were quantified by Western blot and TaqMan™ analysis, respectively. Knock-down of SIM2 was detected at the protein level with SIM2 shRNA sequences 5 and 6 and confirmed by TaqMan™ analysis. Results are depicted in FIG. 2.

The lentiviruses were generated by plating 5-6 million cells in a 10-cm dish and 6 μg of shRNA plasmid for SIM2 (MISSION Sigma; TRCN0000015148 (shRNA1); TRCN0000015149 (shRNA2); TRCN0000015150 (shRNA3); TRCN0000015152 (shRNA4); TRCN0000422875 (shRNA5); TRCN0000429100 (shRNA6)) or scrambled control was transfected with MISSION lentiviral packaging mix (Sigma; SHP001) and Fugene6 (Promega; E2691) following manufacturer's protocol. The resulting lentiviruses were concentrated using Lenti-X concentrator (Takara; 631232) and transduced to HEK293T in the presence of 8 μg/ml polybrene (Sigma; TR-1003-G) and selected with 2 μg/mL puromycin (Gibco; A1113803).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having Parkinson's disease or at risk of developing Parkinson's disease, the method comprising administering a SIM BHLH Transcription Factor 2 (SIM2) agonist to the subject.

2. The method of claim 1, wherein the Parkinson's disease is idiopathic Parkinson's disease, vascular Parkinson's disease, or drug-induced Parkinson's disease.

3-4. (canceled)

5. The method of claim 1, wherein the subject is heterozygous or homozygous for a SIM2 variant nucleic acid molecule.

6. The method of claim 1, wherein the subject is also administered a Parkinson's disease therapeutic agent.

7. The method of claim 6, wherein the Parkinson's disease therapeutic agent comprises carbidopa, levodopa, a combination of carbidopa and levodopa, droxidopa, melevodopa, foscarbidopa, foslevodopa, a dopamine agonist, a monoamine oxidase B (MAO B) inhibitor, a catechol O-methyltransferase (COMT) inhibitor, an anticholinergic, amantadine, an adenosine receptor antagonist, pimavanserin, benserazide, biperiden, bornaprine, quetiapine, rivastigmine, diphenhydramine, or hyoscyamine.

8. The method of claim 7, wherein:
the dopamine agonist comprises pramipexole, rotigotine, apomorphine, bromocriptine, pergolide, ropinirole, piribedil, cabergoline, or lisuride;
the MAO B inhibitor comprises selegiline, rasagiline, or safinamide);
the COMT inhibitor comprises entacapone, tolcapone, or opicapone;
the anticholinergic comprises benztropine or trihexyphenidyl; and
the adenosine receptor antagonist comprises istradefylline.

9. The method of claim 1, further comprising detecting the presence or absence of a SIM2 variant nucleic acid molecule in a biological sample from the subject.

10. The method of claim 9, further comprising administering a Parkinson's disease therapeutic agent in an amount that is the same as a standard dosage amount to the subject when the SIM2 variant nucleic acid molecule is absent from the biological sample.

11. The method of claim 9, further comprising administering a Parkinson's disease therapeutic agent in an amount that is the same as or less than a standard dosage amount to the subject when the subject is heterozygous or homozygous for the SIM2 variant nucleic acid molecule.

12. The method of claim 9, wherein the SIM2 variant nucleic acid molecule comprises a splice-site variant, a stop-gain variant, a start-loss variant, a stop-loss variant, a frameshift variant, a missense variant, an in-frame indel variant, and/or a variant that encodes a truncated SIM2 variant polypeptide.

13. The method of claim 9, wherein the SIM2 variant nucleic acid molecule comprises the genetic variation 21:36719822: TG:T, 21:36726190:C:G, 21:36731044:G:C, 21:36731152: G:A, 21:36741856: T:G, 21:36744979: TG:T, 21:36747923:T:TG, 21:36747923:TG:T, 21:36748041: GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C.

14. The method of claim 1, wherein the SIM2 agonist comprises wild type SIM2 protein or a Cox-2 inhibitor.

15. The method of claim 14, wherein Cox-2 inhibitor comprises celecoxib, rofecoxib, etoricoxib, valdecoxib, or parecoxib.

16. A method of treating a subject having Parkinson's disease or at risk of developing Parkinson's disease by administering a Parkinson's disease therapeutic agent, the method comprising:
determining or having determined whether the subject has a SIM BHLH Transcription Factor 2 (SIM2) variant nucleic acid molecule, by:
obtaining or having obtained a biological sample from the subject; and
performing or having performed a sequence analysis on the biological sample to determine if the subject has a genotype comprising a SIM2 variant nucleic acid molecule; and
administering or continuing to administer the Parkinson's disease therapeutic agent in an amount that is the same as a standard dosage amount to a subject that is SIM2 reference; or
administering or continuing to administer the Parkinson's disease therapeutic agent in an amount that is the same as or less than a standard dosage amount, and/or a SIM2 agonist to a subject that is heterozygous or homozygous for the SIM2 variant nucleic acid molecule;
wherein the presence of the SIM2 variant nucleic acid molecule indicates the subject has an increased risk of developing Parkinson's disease.

17. The method of claim 16, wherein the subject is heterozygous or homozygous for the SIM2 variant nucleic acid molecule, and the subject is administered or continued to be administered the Parkinson's disease therapeutic agent in an amount that is the same as or less than a standard dosage amount and the SIM2 agonist.

18. The method of claim 16, wherein the subject is SIM2 reference, and the subject is administered or continued to be administered the Parkinson's disease therapeutic agent in an amount that is the same a standard dosage amount.

19. The method of claim 16, wherein the SIM2 variant nucleic acid molecule comprises a splice-site variant, a stop-gain variant, a start-loss variant, a stop-loss variant, a frameshift variant, a missense variant, an in-frame indel variant, and/or a variant that encodes a truncated SIM2 variant polypeptide.

20. The method of claim 16, wherein the SIM2 variant nucleic acid molecule comprises the genetic variation 21:36719822:TG:T, 21:36726190:C:G, 21:36731044:G:C, 21:36731152:G:A, 21:36741856:T:G, 21:36744979:TG:T, 21:36747923:T:TG, 21:36747923:TG:T, 21:36748041:GC:G, 21:36911797:G:A, 21:36876382:A:G, 21:36888743:C:A, 21:36865200:A:G, 21:36960371:C:T (rs412162), 21:37007021:C:T (rs10432862), 21:36989046:A:T (rs185763474), 21:37004264:G:A, 21:37031534:G:A, or 21:37022805:T:C.

21. The method of claim 16, wherein the Parkinson's disease therapeutic agent comprises carbidopa, levodopa, a combination of carbidopa and levodopa, droxidopa, melevodopa, foscarbidopa, foslevodopa, a dopamine agonist, a monoamine oxidase B (MAO B) inhibitor, a catechol O-methyltransferase (COMT) inhibitor, an anticholinergic, amantadine, an adenosine receptor antagonist, pimavanserin, benserazide, biperiden, bornaprine, quetiapine, rivastigmine, diphenhydramine, or hyoscyamine.

22. The method of claim 21, wherein:
the dopamine agonist comprises pramipexole, rotigotine, apomorphine, bromocriptine, pergolide, ropinirole, piribedil, cabergoline, or lisuride;
the MAO B inhibitor comprises selegiline, rasagiline, or safinamide);
the COMT inhibitor comprises entacapone, tolcapone, or opicapone;
the anticholinergic comprises benztropine or trihexyphenidyl; and
the adenosine receptor antagonist comprises istradefylline.

23. The method of claim 16, wherein the SIM2 agonist comprises wild type SIM2 protein or a Cox-2 inhibitor.

24. The method of claim 23, wherein Cox-2 inhibitor comprises celecoxib, rofecoxib, etoricoxib, valdecoxib, or parecoxib.

25. A method of identifying a subject having an increased risk of developing Parkinson's disease, the method comprising:
determining or having determined the presence or absence of a SIM BHLH Transcription Factor 2 (SIM2) variant nucleic acid molecule in a biological sample obtained from the subject;
wherein:
when the subject is SIM2 reference, then the subject has a decreased risk of developing Parkinson's disease; and
when the subject is heterozygous or homozygous for the SIM2 variant nucleic acid molecule, then the subject has an increased risk of developing Parkinson's disease.

26-44. (canceled)
